(19) United States
(12) Patent Application Publication
(10) Pub. No.: US 2026/0294464 A1
(43) Pub. Date: Oct. 1, 2026

(54) SYSTEM AND METHOD FOR CONTROLLED ULTRASONIC SEALING AND CUTTING

(71) Applicant: INTUITIVE SURGICAL OPERATIONS, INC., Sunnyvale, CA (US)

(72) Inventors: Jignesh M. SHAH, Sunnyvale, CA (US); Iman KHALAJI, San Jose, CA (US); Duane W. MARION, Scottsdale, AZ (US)

(21) Appl. No.: 19/477,868

(22) PCT Filed: Apr. 23, 2024
(86) PCT No.: PCT/US2024/025800

§ 371 Date: Oct. 23, 2025

Related U.S. Application Data
(60) Provisional application No. 63/498,465, filed on Apr. 26, 2023.

(51) Int. Cl. A61B 17/32; A61B 17/00; A61B 18/00; A61B 18/14

(52) U.S. Cl. CPC A61B 17/320092; A61B 18/1445; A61B 2017/00026; A61B 2017/320094; A61B 2017/320095; A61B 2018/00601; A61B 2018/0063

(57) ABSTRACT

In various embodiments, a computer-assisted device comprising an end effector having a first jaw, a second jaw, and at least one transducer for delivering ultrasonic energy using at least one of the first jaw or the second jaw, and a processing system coupled to the end effector, the processing system being configured to control the first jaw and the second jaw to grasp a material, determine one or more first characteristics of the grasping, control a first energy delivered by the at least one transducer to seal the material based on the one or more first characteristics, and in response to completing sealing of the material, control at least a second energy delivered by the at least one transducer to cut the grasped material based on the one or more first characteristics.

### RELATED APPLICATIONS

This application claims the benefit to U.S. Provisional Application No. 63/498,465, filed Apr. 26, 2023, and entitled “System and Method for Controlled Ultrasonic Sealing and Cutting,” the subject matter of which is incorporated by reference herein.

### TECHNICAL FIELD

The present disclosure relates generally to computer-assisted systems and more particularly to a system and method for controlled ultrasonic sealing and cutting.

### BACKGROUND

More and more devices are being replaced with computer-assisted electronic devices. This is especially true in industrial, entertainment, educational, and other settings. As a medical example, the hospitals of today with large arrays of electronic devices being found in operating rooms, interventional suites, intensive care wards, emergency rooms, and/or the like. For example, glass and mercury thermometers are being replaced with electronic thermometers, intravenous drip lines now include electronic monitors and flow regulators, and traditional hand-held surgical and other medical instruments are being replaced by computer-assisted medical devices.

These computer-assisted devices are useful for performing operations and/or procedures on materials, such as the soft or hard tissue of a patient. With many computer-assisted devices, an operator, such as a surgeon and/or other medical personnel, can typically manipulate input devices using one or more controls on an operator console. As the operator operates the various controls at the operator console, the commands are relayed from the operator console to a computer-assisted device located in a workspace where they are used to position and/or actuate one or more end effectors and/or instruments that are mounted (e.g., via repositionable arms) to the computer-assisted device. In this way, the operator is able to perform one or more procedures on material in the workspace using the end effectors and/or instruments. Depending upon the desired procedure and/or the instruments in use, the desired procedure can be performed partially or wholly under control of the operator using teleoperation and/or under semi-autonomous control where the computer-assisted device can perform a sequence of operations based on one or more activation actions by the operator.

Computer-assisted devices, whether actuated manually, teleoperatively, and/or semi-autonomously can be used in a variety of operations and/or procedures and can have various configurations. Many such instruments include an end effector mounted at a distal end of a shaft that can be mounted to the distal end of a repositionable or articulated arm. In many operational scenarios, the shaft can be configured to be inserted into the workspace via an opening in the workspace. As a medical example, the shaft can be inserted (e.g., laparoscopically, thoracoscopically, and/or the like) through an opening (e.g., a body wall incision, a natural orifice, and/or the like) to reach a remote surgical site. In some instruments, an articulating wrist mechanism can be mounted to the distal end of the instrument's shaft to support the end effector with the articulating wrist providing the ability to alter an orientation of the end effector relative to a longitudinal axis of the shaft.

End effectors of different design and/or configuration can be used to perform different tasks, procedures, and functions so as to allow the operator to perform any of a variety of procedures on a material. Examples include, but are not limited to, cauterizing, ablating, suturing, cutting, stapling, fusing, sealing, etc., and/or combinations thereof. Accordingly, end effectors can include a variety of components and/or combinations of components to perform these procedures.

In many embodiments, the size of the end effector is typically kept as small as possible while still allowing it to perform its intended task. One approach to keeping the size of the end effector small is to accomplish actuation of the end effector through the use of one or more inputs at a proximal end of the instrument, which is typically located externally and/or peripherally to the workspace. Various gears, levers, pulleys, cables, rods, bands, and/or the like, can then be used to transmit actions from the one or more inputs along the shaft of the instrument and to actuate the end effector. In some embodiments, a transmission mechanism at the proximal end of the instrument interfaces with various motors, solenoids, servos, active actuators, hydraulics, pneumatics, and/or the like provided on a repositionable arm of the computer-assisted device. The motors, solenoids, servos, active actuators, hydraulics, pneumatics, and/or the like typically receive control signals through a controller and provide input in the form of force and/or torque at the proximal end of the transmission mechanism, which the various gears, levers, pulleys, cables, rods, bands, and/or the like ultimately transmit to actuate the end effector at the distal end of the transmission mechanism.

Additionally, in many embodiments, the instruments and/or end effectors can include one or more energy delivery components that can be used to deliver ultrasonic, radio frequency, electrical, magnetic, thermal, light, and/or other energies to the material grasped by and/or in proximity to the end effector. In some embodiments, the end effectors can include one or more sensors for monitoring the energy delivery. Various wires, cables, optical fibers, and/or like can be used to deliver the energy to end effector from a control module located proximal to the end effector (e.g., in a control console) and/or provide the sensor information to the control module.

When delivering energy via the end effector, various kinematic controls, such as griping and grasping, and energy controls, such as cutting and sealing, are independently operated. For example, during ultrasonic vessel sealing, a user controls the jaws of an end effector to grasp a portion of tissue. The user then applies ultrasonic energy to the grasped material to seal and cut the material.

Accordingly, improved methods and systems for controlled ultrasonic sealing, such as computer-assisted devices having end effectors used to grasp and/or deliver ultrasonic energy, are desirable. In some examples, it can be desirable to provide control of the computer-assisted device and/or the end effectors to enact synchronous sealing and cutting of material through transfer of ultrasonic energy so as to help ensure that the instrument is able to successfully perform a desired procedure on the material.

### SUMMARY

Consistent with some embodiments, a computer-assisted system, and method implemented therein, includes a computer-assisted device comprising an end effector having a first jaw, a second jaw, and at least one transducer for delivering ultrasonic energy using at least one of the first jaw or the second jaw, and processing system coupled to the end effector, the processing system being configured to control the first jaw and the second jaw to grasp a material, determine one or more first characteristics of the grasping, control a first energy delivered by the at least one transducer to seal the material based on the one or more first characteristics, and in response to completing sealing of the material, control at least a second energy delivered by the at least one transducer to cut the grasped material based on the one or more first characteristics.

Consistent with some embodiments, one or more non-transitory machine-readable media include a plurality of machine-readable instructions which when executed by a processor system are adapted to cause the processor system to perform any of the methods described herein.

### BRIEF DESCRIPTION OF THE DRAWINGS

So that the manner in which the above recited features of the various embodiments can be understood in detail, a more particular description of the inventive concepts, briefly summarized above, can be had by reference to various embodiments, some of which are illustrated in the appended drawings. It is to be noted, however, that the appended drawings illustrate only typical embodiments of the inventive concepts and are therefore not to be considered limiting of scope in any way, and that there are other equally effective embodiments.

FIG. 1 is diagram of a computer-assisted system in accordance with one or more embodiments.

FIG. 2 is diagram of a computer-assisted system in accordance with one or more embodiments.

FIG. 3 is a simplified diagram showing an instrument suitable for use with the computer-assisted system of FIG. 1 in accordance with one or more embodiments.

FIG. 4 depicts an effector having a first and second jaw and illustrate jaw characteristics in accordance with one or more embodiments.

FIG. 5 depicts another effector having a first and second jaw and illustrate the jaw characteristics in accordance with one or more embodiments.

FIG. 6 illustrate the user interface having an indicator of sealing status in accordance with one or more embodiments.

FIG. 7 is a flow diagram of a method for ultrasonic energy delivery in accordance with one or more embodiments.

In the figures, elements having the same designations have the same or similar functions.

### DETAILED DESCRIPTION

In this description, specific details are set forth describing some embodiments consistent with the present disclosure. Numerous specific details are set forth in order to provide a thorough understanding of the embodiments. It will be apparent, however, to one skilled in the art that some embodiments can be practiced without some or all of these specific details. The specific embodiments disclosed herein are meant to be illustrative but not limiting. One skilled in the art can realize other elements that, although not specifically described here, are within the scope and the spirit of this disclosure. In addition, to avoid unnecessary repetition, one or more features shown and described in association with one embodiment can be incorporated into other embodiments unless specifically described otherwise or if the one or more features would make an embodiment non-functional.

Further, the terminology in this description is not intended to limit the invention. For example, spatially relative terms—such as “beneath”, “below”, “lower”, “above”, “upper”, “proximal”, “distal”, and the like-can be used to describe the relation of one element or feature to another element or feature as illustrated in the figures. These spatially relative terms are intended to encompass different positions (i.e., locations) and orientations (i.e., rotational placements) of the elements or their operation in addition to the position and orientation shown in the figures. For example, if the content of one of the figures is turned over, elements described as “below” or “beneath” other elements or features would then be “above” or “over” the other elements or features. A device can be otherwise oriented and the spatially relative descriptors used herein interpreted accordingly. Likewise, descriptions of movement along and around various axes include various special element positions and orientations. In addition, the singular forms “a”, “an”, and “the” are intended to include the plural forms as well, unless the context indicates otherwise. And, the terms “comprises”, “comprising”, “includes”, and the like specify the presence of stated features, steps, operations, elements, and/or components but do not preclude the presence or addition of one or more other features, steps, operations, elements, components, and/or groups. Components described as coupled can be electrically or mechanically directly coupled, or they can be indirectly coupled via one or more intermediate components.

Elements described in detail with reference to one embodiment, implementation, or module can, whenever practical, be included in other embodiments, implementations, or modules in which they are not specifically shown or described. For example, if an element is described in detail with reference to one embodiment and is not described with reference to a second embodiment, the element can nevertheless be claimed as included in the second embodiment. Thus, to avoid unnecessary repetition in the following description, one or more elements shown and described in association with one embodiment, implementation, or application can be incorporated into other embodiments, implementations, or aspects unless specifically described otherwise, unless the one or more elements would make an embodiment or implementation non-functional, or unless two or more of the elements provide conflicting functions.

In some instances, well known methods, procedures, components, and circuits have not been described in detail so as not to unnecessarily obscure aspects of the embodiments.

This disclosure describes various devices, elements, and portions of computer-assisted systems and elements in terms of their state in three-dimensional space. As used herein, the term “position” refers to the location of an element or a portion of an element (e.g., three degrees of translational freedom in a three-dimensional space, such as along Cartesian x-, y-, and z-coordinates). As used herein, the term “orientation” refers to the rotational placement of an element or a portion of an element (e.g., three degrees of rotational freedom in three-dimensional space, such as about roll, pitch, and yaw axes, represented in angle-axis, rotation matrix, quaternion representation, and/or the like). As used herein, and for a device with a kinematic series, such as with a repositionable structure with a plurality of links coupled by one or more joints, the term “proximal” refers to a direction toward a base of the kinematic series, and “distal” refers to a direction away from the base along the kinematic series.

As used herein, the term “pose” refers to the multi-degree of freedom (DOF) spatial position and orientation of a coordinate system of interest attached to a rigid body. In general, a pose includes a pose variable for each of the DOFs in the pose. For example, a full 6-DOF pose for a rigid body in three-dimensional space would include 6 pose variables corresponding to the 3 positional DOFs (e.g., x, y, and z) and the 3 orientational DOFs (e.g., roll, pitch, and yaw). A 3-DOF position only pose would include only pose variables for the 3 positional DOFs. Similarly, a 3-DOF orientation only pose would include only pose variables for the 3 rotational DOFs. Further, a velocity of the pose captures the change in pose over time (e.g., a first derivative of the pose). For a full 6-DOF pose of a rigid body in three-dimensional space, the velocity would include 3 translational velocities and 3 rotational velocities. Poses with other numbers of DOFs would have a corresponding number of velocities translational and/or rotational velocities.

Aspects of this disclosure are described in reference to computer-assisted systems, which can include devices that are teleoperated, externally manipulated, autonomous, semiautonomous, and/or the like. Further, aspects of this disclosure are described in terms of an implementation using a teleoperated surgical system, such as the da Vinci® Surgical System commercialized by Intuitive Surgical, Inc. of Sunnyvale, California. Knowledgeable persons will understand, however, that inventive aspects disclosed herein can be embodied and implemented in various ways, including teleoperated and non-teleoperated, and medical and non-medical embodiments and implementations. Implementations on da Vinci® Surgical Systems are merely exemplary and are not to be considered as limiting the scope of the inventive aspects disclosed herein. For example, techniques described with reference to surgical instruments and surgical methods can be used in other contexts. Thus, the instruments, systems, and methods described herein can be used for humans, animals, portions of human or animal anatomy, industrial systems, general robotic, or teleoperated systems. As further examples, the instruments, systems, and methods described herein can be used for non-medical purposes including industrial uses, general robotic uses, sensing or manipulating non-tissue work pieces, cosmetic improvements, imaging of human or animal anatomy, gathering data from human or animal anatomy, setting up or taking down systems, training medical or non-medical personnel, and/or the like. Additional example applications include use for procedures on tissue removed from human or animal anatomies (with or without return to a human or animal anatomy) and for procedures on human or animal cadavers. Further, these techniques can also be used for medical treatment or diagnosis procedures that include, or do not include, surgical aspects.

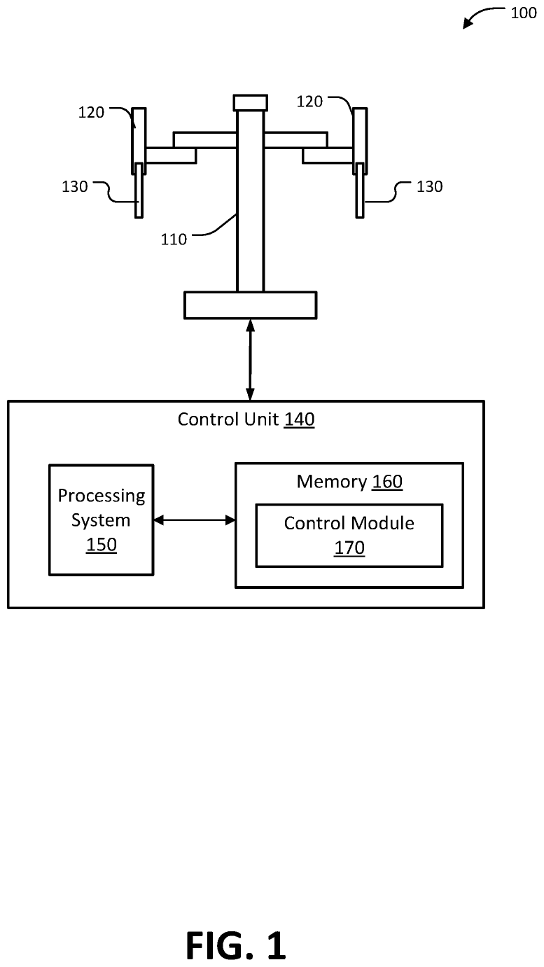

FIG. 1 is diagram of a computer-assisted system 100 in accordance with one or more embodiments. As shown in FIG. 1, the computer-assisted system 100 includes, without limitation, a manipulating assembly 110 with one or more repositionable structures 120 and one or more instruments 130. The computer-assisted system 100 also includes, without limitation, a control unit including a processing system 150, memory 160, and a control module 170.

In the example of FIG. 1, the repositionable structure(s) 120 are shown as manipulator arms comprising a plurality of links coupled by one or more joints. Each of the one or more repositionable structures 120 supports one or more instruments 130. In some examples, the manipulating assembly 110 comprises a computer-assisted surgical assembly. Examples of medical instruments include surgical instruments for interacting with tissue, imaging, sensing devices, and/or the like. In some examples, the instruments 130 can include end effectors that are capable of, but are not limited to, performing, gripping, retracting, cauterizing, ablating, suturing, cutting, stapling, fusing, sealing, etc., and/or combinations thereof.

In a teleoperation example, the manipulating assembly 110 can further be communicatively coupled by wired or wireless connection to a user input system (not shown). The user input system includes one or more input controls for operating the manipulating assembly 110, the one or more repositionable structures 120, and/or the instruments 130. In some examples, the one or more input controls can include kinematic series of links and one or more joint(s), one or more actuators for driving portions of the input control(s), robotic manipulators, levers, pedals, switches, keys, knobs, triggers, and/or the like. In some examples, the one or more input controls comprise a leader device (also called a “master” device in industry), and the manipulating assembly 110 and/or the one or more repositionable structures 120 (either supporting or not supporting instruments 130) comprise a follower device (also called a “slave” device in industry). An operator can use the one or more input controls to command motion of the manipulating assembly 110, such as by commanding motion of the one or more repositionable structures 120 and/or instruments 130, in a leader-follower configuration. The leader-follower configuration is a type of teleoperation configuration, and is sometimes called a master-slave configuration in industry.

The manipulating assembly 110 of FIG. 1 is coupled to a control unit 140 via an interface. The interface can be wired and/or wireless, and can include one or more cables, fibers, connectors, and/or buses and can further include one or more networks with one or more network switching and/or routing devices. Operation of the control unit 140 is controlled by a processing system 150. The processing system 150 can include one or more central processing units, multi-core processors, microprocessors, microcontrollers, digital signal processors, field programmable gate arrays (FPGAs), application specific integrated circuits (ASICs), graphics processing units (GPUs), tensor processing units (TPUs), and/or the like in the control unit 140. The control unit 140 can be implemented as a stand-alone subsystem and/or board added to a computing device or as a virtual machine. In some embodiments, the control unit 140 can be included as part of the user input system and/or the manipulating assembly 110, and/or be operated separately from, and in coordination with, the user input system and/or the manipulating assembly 110.

As one example, the manipulating assembly 110, the user input system, and/or the control unit 140 can correspond to the patient side cart, the surgeon console, and the processing units and associated software of da Vinci® Surgical System commercialized by Intuitive Surgical, Inc. of Sunnyvale, California. In some embodiments, manipulating assemblies with other configurations, such as fewer or more repositionable structures, different user input systems or input controls, different repositionable structure hardware, and/or the like, can comprise the computer-assisted system 100.

The memory 160 can be used to store software executed by the control unit 140 and/or one or more data structures used during operation of the control unit 140. The memory 160 can include one or more types of machine-readable media. Some common forms of machine-readable media can include floppy disk, flexible disk, hard disk, magnetic tape, any other magnetic medium, CD-ROM, any other optical medium, punch cards, paper tape, any other physical medium with patterns of holes, RAM, PROM, EPROM, FLASH-EPROM, any other memory chip, or cartridge, and/or any other medium from which a processor or computer is adapted to read.

As shown in the example of FIG. 1, the memory 160 includes a control module 170 that can be used to support autonomous, semiautonomous, and/or teleoperated control of the manipulating assembly 110. The control module 170 includes one or more application programming interfaces (APIs) for receiving position, motion, force, torque, and/or other sensor information from the manipulating assembly 110, the repositionable structures 120, and/or the instruments 130, for sharing position, motion, force, torque, and/or collision avoidance information with other control units regarding other devices, and/or planning and/or assisting in the planning of motion for the manipulating assembly 110 (such as motion of the repositionable structures 120), and/or the instruments 130. In some examples, the control module 170 further supports autonomous, semiautonomous, and/or teleoperated control of the manipulating assembly 110 and/or the instruments 130 during the performance of various tasks. And although the control module 170 is depicted as a software application, the control module 170 can optionally be implemented using hardware, software, and/or a combination of hardware and software.

In various embodiments, the control module 170 is responsible for managing the mechanical operation and/or energy delivery operation of the one or more instruments 130. In some examples, the control module 170 monitors one or more sensors (e.g., one or more encoders, potentiometers, fiber optic sensors, and/or the like) used to track the position, orientation, articulation, and/or mechanical actuation of the one or more instruments 130 and their respective end effectors and/or one or more material properties of material being interacted with by the one or more instruments 130 and their respective end effectors. In some examples, the control module 170 controls the position, orientation, articulation, and/or mechanical actuation of the one or more instruments 130 and their respective end effectors using one or more actuators based on the monitoring. In some examples, control of the position, orientation, articulation, and/or mechanical actuation of the one or more instruments 130 and their respective end effectors includes controlling one or more degrees of freedom including, as examples, an insertion depth, a roll, a pitch, a yaw, a wrist articulation, an angle between jaws, a separation distance between jaws, a force or torque applied, an amount of sealing, cutting, and/or transection using a moveable element, an amount of stapling, and/or the like.

In various embodiments, the control module 170 is responsible for managing the energy delivery operations of the one or more instruments 130. In some examples, the control module 170 monitors one or more sensors used to track the energy delivered by the one or more instruments 130 and their respective end effectors and/or one or more material properties of material being interacted with by the one or more instruments 130 and their respective end effectors. In some examples, the control module 170 controls the amount of ultrasonic energy delivered by the one or more instruments 130 and their respective end effectors using one or more transducers, signal generators, and/or the like based on the monitoring.

In some embodiments, the control module 170 includes one or more models (e.g., various prediction models, material models, kinematic models, and/or other deep learning models) that are used by the control module 170 to control mechanical movement and/or energy delivery of the one or more instruments 130 and their respective end effectors. In some examples, the one or more models provide recommendations regarding mechanical movement and/or energy delivery by the one or more instruments 130 and their respective end effectors as is described in further detail below. For example, the control module 170 can use the one or more models to set threshold values associated with sealing and/or cutting operations using ultrasonic energy. In some examples, the one or more models include one or more functions, one or more look up tables, one or more maps, one or more parameterized curves, one or more machine learning models (e.g., one or more neural networks), and/or the like. In some examples, the one or more parameterized curves include linear relationships, piece-wise linear relationships, quadratic relationships, higher-order relationships, and/or the like determined via curve fitting, regression, and/or the like from data collected from previous grasp and/or energy delivery applications.

As discussed above and further emphasized here, FIG. 1 is merely an example which should not unduly limit the scope of the claims. One of ordinary skill in the art would recognize many variations, alternatives, and modifications. According to some embodiments, computer-assisted system 100 can include any number of computer-assisted devices with articulated arms and/or instruments of similar and/or different in design from computer-assisted device 110. In some examples, each of the computer-assisted devices can include fewer or more articulated arms and/or instruments. In some medical embodiments, the computer-assisted system 100 can be found in a clinic, diagnostic facility, an operating room, an interventional suite, or other medical environment. Although the computer-assisted system 100 is shown comprising one manipulating assembly 110 with two repositionable structures 120, each supporting a corresponding instrument 130, one of ordinary skill would understand that the computer-assisted system 100 can include any number of manipulating assemblies, each manipulating assembly can comprise one or more repositionable structures, and each repositionable structure can support one or more instruments, and that all of these elements can be similar or different in design from that specifically depicted in these figures. In some examples, each of the manipulating assemblies can include fewer or more repositionable structures, and/or support fewer or more instruments, than specifically depicted in these figures.

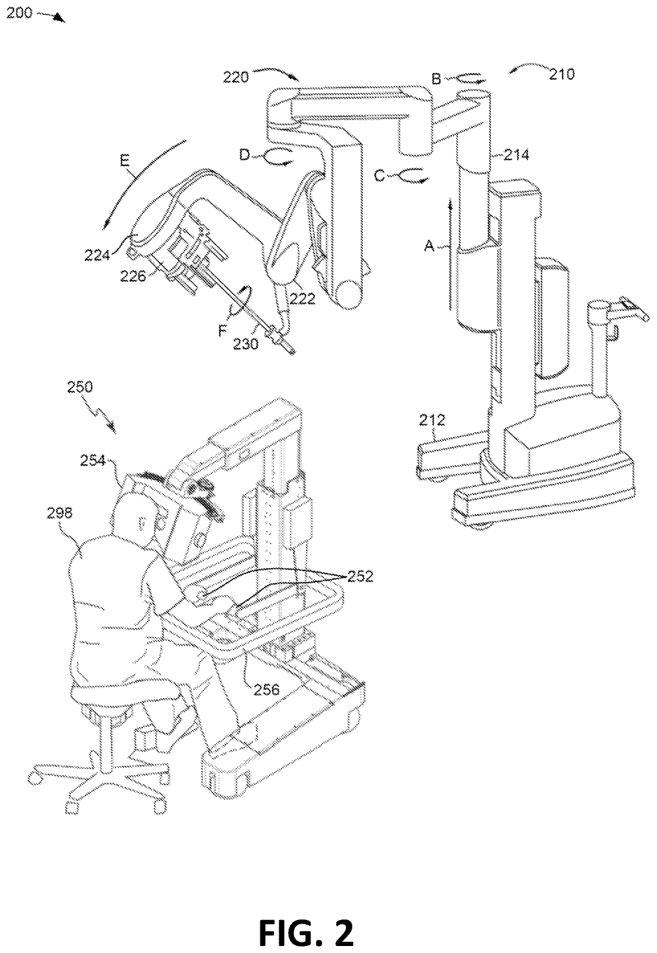

FIG. 2 is diagram of a computer-assisted system in accordance with one or more embodiments. The computer-assisted system 200, in the example of FIG. 2, includes a repositionable structure shown as a manipulating assembly 210, and a user input system 250. In a teleoperation scenario, an operator 298 uses the user input system 250 to operate the manipulating assembly 210, such as in a leader-follower configuration. In the leader-follower configuration of FIG. 2, a component of the user input system 250 (e.g., an input control) is the leader, and a portion of the manipulating assembly 210 (e.g., a manipulator arm or other repositionable structure) is the follower.

The manipulating assembly 210 can be used to introduce a set of instruments into a work site through a single port 230 (e.g., using a cannula as shown) inserted in an aperture. In a medical scenario, the work site can be on or within a body of a patient, and the aperture can be a minimally invasive incision or a natural body orifice. The port 230 can be free-floating, held in place by a fixture separate from the manipulating assembly 210, or held by a linkage 222 or other part of the manipulating assembly 210. The linkage 222 can be coupled to additional joints and links 214, 220 of the manipulating assembly 210, and these additional joints and links 214, 220 can be mounted on a base 212. The linkage 222 can further include a manipulator-supporting link 224 located in a proximal direction 262 to the port 230. A set of manipulators 226 located in the proximal direction 262 to the port 230 can couple to the manipulator-supporting link 224. The repositionable structure that can be moved to follow commands from the user input system 250 can include one or more of any of the following: the linkage 222, additional joints and links 214, 220, base 212, manipulator-supporting link 224, and/or any additional links or joints coupled to the foregoing joints or links. Each of the manipulators 226 can include a carriage (or other instrument-coupling link) configured to couple to an instrument, and each of the manipulators 226 can include one or more joint(s) and/or link(s) that can be driven to move the carriage. For example, a manipulator 226 can include a prismatic joint that, when driven, linearly moves the carriage and any instrument(s) coupled to the carriage. This linear motion can be along (parallel to) an insertion axis that extends in a distal direction 264 to and through port 230.

The additional joints and additional links 214, 220 can be used to position the port 230 at the aperture or another position. FIG. 2 shows a prismatic joint for vertical adjustment (as indicated by arrow “A”) and a set of rotary joints for horizontal adjustment (as indicated by arrows “B” and “C”) that can be used to translate a position of the port 230. The linkage 222 is used to pivot the port 230 (and the instruments disposed within the port at the time) in yaw, pitch, and roll angular rotations about a remote center of motion (RCM) located in proximity to port 230 as indicated by arrows D, E, and F, respectively, without translating the RCM.

Actuation of the degrees of freedom provided by joint(s) of the instrument(s) (not shown) can be provided by actuators disposed in, or whose motive force (e.g., linear force or rotary torque) is transmitted to, the instrument(s). Examples of actuators include rotary motors, linear motors, solenoids, and/or the like. The actuators can drive transmission elements in the manipulating assembly 210 and/or in the instruments to control the degrees of freedom of the instrument(s). For example, the actuators can drive rotary discs of the manipulator that couple with drive elements (e.g., rotary discs, linear slides) of the instrument(s), where driving the driving elements of the instruments drives transmission elements in the instrument that couple to move the joint(s) of the instrument, or to actuate some other function of the instrument, such as a degree of freedom of an end effector. Accordingly, the degrees of freedom of the instrument(s) can be controlled by actuators that drive the instrument(s) in accordance with control signals. The control signals can be determined to cause instrument motion or other actuation as determined automatically by the system, as indicated to be commanded by movement or other manipulation of the input controls, or any other control signal.

Furthermore, appropriately positioned sensors, e.g., encoders, potentiometers, and/or the like, can be provided to enable measurement of indications of the joint positions, or other data that can be used to derive joint position, such as joint velocity. The actuators and sensors can be disposed in, or transmit to or receive signals from, the manipulator(s) 226. Techniques for manipulating multiple instruments in a computer-assisted system are described more fully in Patent Cooperation Treaty Patent Application No. PCT/US2021/0473506, filed Aug. 24, 2021, and entitled “METHOD AND SYSTEM FOR COORDINATED MULTIPLE-TOOL MOVEMENT USING A DRIVABLE ASSEMBLY,” which is incorporated herein by reference.

While a particular configuration of the manipulating assembly 210 is shown in FIG. 2, those skilled in the art will appreciate that embodiments of this disclosure can be used with any design of manipulating assembly or other repositionable structure. In some examples, a manipulating assembly can have any number and any types of degrees of freedom, can be configured to couple or not couple to an entry port, can optionally use a port other than a cannula, such as a guide tube, and/or the like. In some examples, the manipulating assembly 210 can also include an arrangement of links and joints that does not provide a remote center of motion.

In the example shown in FIG. 2, the user input system 250 includes one or more input controls 252 configured to be operated by the operator 298. In the example shown in FIG. 2, the one or more input controls 252 are contacted and manipulated by the hands of the operator 298, with one input control 252 for each hand. Examples of such hand-input-devices include any type of device manually operable by human user, e.g., joysticks, trackballs, button clusters, and/or other types of haptic devices typically equipped with multiple degrees of freedom. Position, force, and/or tactile feedback devices (not shown) can be employed to transmit position, force, and/or tactile sensations from the instruments back to the hands of the operator 298 through the input controls 252.

The input controls 252 are supported by the user input system 250 and are shown as mechanically grounded, and in other implementations can be mechanically ungrounded. An ergonomic support 256 can be provided in some implementations; for example, FIG. 2 shows an ergonomic support 256 including forearm rests on which the operator 298 can rest his or her forearms while manipulating the input controls 252. In some examples, the operator 298 can perform tasks at a work site near the manipulating assembly 210 during a procedure by controlling the manipulating assembly 210 using the input controls 252.

A display unit 254 is included in the user input system 250. The display unit 254 can display images for viewing by the operator 298. The display unit 254 can provide the operator 298 with a view of the worksite with which the manipulating assembly 210 interacts. The view can include stereoscopic images or three-dimensional images to provide a depth perception of the worksite and the instrument(s) of the manipulating assembly 210 in the worksite. The display unit 254 can be moved in various degrees of freedom to accommodate the viewing position of the operator 298 and/or to provide control functions. Where a display unit (such as the display unit 254 is also used to provide control functions, such as to command the manipulating assembly 210, the display unit also includes an input control (e.g., another input control 252).

When using the user input system 250, the operator 298 can sit in a chair or other support, position his or her eyes to see images displayed by the display unit 254, grasp and manipulate the input controls 252, and rest his or her forearms on the ergonomic support 256 as desired. In some implementations, the operator 298 can stand at the station or assume other poses, and the display unit 254 and input controls 252 can differ in construction, be adjusted in position (height, depth, etc.), and/or the like.

In some examples, the repositionable structure includes a base manipulator and multiple instrument manipulators coupled to the base manipulator. In some examples, the repositionable structure includes a single instrument manipulator and no serial coupling of manipulators. In some examples, the repositionable structure includes a single instrument manipulator coupled to a single base manipulator. In some examples, the computer-assisted system can include a moveable-base that is cart-mounted or mounted to an operating table, and one or more manipulators mounted to the moveable base.

In many of the examples described in this application, the repositionable structure includes one or more proximal repositionable structures and one or more distal repositionable structures. In some examples, the one or more proximal repositionable structures can include one or more of any of the linkage 222, additional joints and/or links 214, 220, manipulator-supporting link 224, and/or any additional links and/or joints coupled to the foregoing joints or links. The one or more distal repositionable structures can include one or more of the manipulators 226, carriages (or other instrument-coupling links) configured to couple to instruments, and/or one or more joint(s) and/or link(s) that can be driven to move the carriages. The operator 298 views the workspace via an imaging device coupled to one or more distal repositionable structure(s) in the form of one of the manipulators 226 and associated carriages, joints, and/or links. The imaging device has a field of view that can be displayed on the display unit 254. The operator 298 contacts and manipulates the one or more input controls 252 to generate commanded motions to move the repositionable structure. In response, one or more corresponding distal repositionable structures move in order to move the instruments according to the commanded motion.

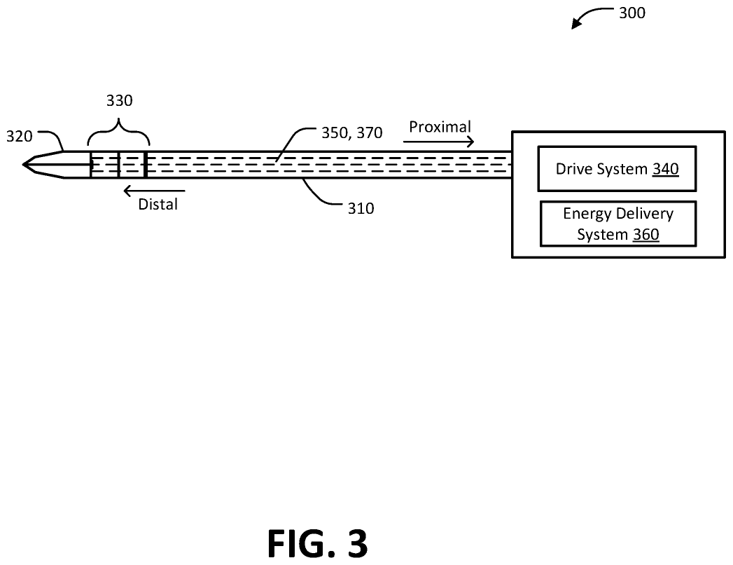

FIG. 3 is a simplified diagram showing an instrument 300 suitable for use with the computer-assisted system of FIG. 1 in accordance with one or more embodiments. In some embodiments, the instrument 300 is consistent with any of the instruments 130 of FIG. 1. The directions “proximal” and “distal” as depicted in FIG. 3 and as used herein help describe the relative orientation and location of components of the instrument 300.

As shown in FIG. 3, the instrument 300 includes a long shaft 310 used to couple an end effector 320 located at a distal end of the shaft 310 to where the instrument 300 is mounted to a repositionable arm and/or a computer-assisted device at a proximal end of the shaft 310. Depending upon the particular procedure for which the instrument 300 is being used, the shaft 310 can be inserted through an opening (e.g., a body wall incision, a natural orifice, and/or the like) in order to place the end effector 320 in proximity to a workspace, such as a remote surgical site located within the anatomy of a patient. As further shown in FIG. 3, the end effector 320 is generally consistent with a two-jawed gripper-style end effector, which in some embodiments can further include an energy delivery system 360. However, one of ordinary skill would understand that different instruments 300 with different end effectors 320 are possible and can be consistent with the embodiments of the instrument 300 as described elsewhere herein.

An instrument, such as the instrument 300 with the end effector 320, typically relies on multiple degrees of freedom (DOFs) during its operation. Depending upon the configuration of the instrument 300 and the repositionable arm and/or computer-assisted device to which it is mounted, various DOFs that can be used to position, orient, and/or operate the end effector 320 are possible. In some examples, the shaft 310 can be inserted in a distal direction and/or retreated in a proximal direction to provide an insertion DOF that can be used to control how deep within the workspace the end effector 320 is placed. In some examples, the shaft 310 can be able rotate about its longitudinal axis to provide a roll DOF that can be used to rotate the end effector 320. In some examples, additional flexibility in the position and/or orientation of the end effector 320 can be provided by an articulated wrist 330 that is used to couple the end effector 320 to the distal end of the shaft 310.

In some examples, the articulated wrist 330 can include one or more rotational joints, such as one or more roll, pitch or yaw joints that can provide one or more “roll,” “pitch,” and “yaw” DOF(s), respectively, that can be used to control an orientation of the end effector 320 relative to the longitudinal axis of the shaft 310. In some examples, the one or more rotational joints can include a pitch and a yaw joint; a roll, a pitch, and a yaw joint, a roll, a pitch, and a roll joint; and/or the like. In some examples, the articulated wrist 330 can include one or more transducers that converts electrical energy received from the energy delivery system 360 into mechanical motion (e.g., providing ultrasonic energy by vibrating one or more portions of the end effector 320).

In some examples, the end effector 320 can further include a grip DOF used to control the opening and closing of the jaws of the end effector 320. Depending upon the configuration, the end effector 320 can include two moveable jaws that are articulated with respect to each other about a hinge point located near a proximal end of the end effector 320, one fixed jaw and one moveable jaw that is articulated with respect to the fixed jaw about the hinge point, one fixed jaw and one moveable jaw that is articulated with respect to the fixed jaw along a track (e.g., clamp jaw) and/or two moveable jaws that are articulated with respect to each other about a track. In some examples, the two moveable jaws can include two parallel jaw faces whose distance there between is adjusted, such as by using one or more cams, to open and close the jaws. In some examples, each jaw face is generally planar and is parallel with a jaw face of an opposing jaw when the jaw 402 closed relative to the opposing jaw, and at an angle to the jaw face of the opposing jaw when the jaw is open relative to the opposing jaw. In some examples, the jaw face is curved (e.g., curving downwards at an end distal to the articulated wrist 330). In such instances, the opposing jaw matches the curvature of the jaw face when closed.

The instrument 300 further includes a drive system 340 located at the proximal end of the shaft 310. The drive system 340 includes one or more components for introducing forces and/or torques to the instrument 300 that can be used to manipulate the various DOFs supported by the instrument 300. In some examples, the drive system 340 includes one or more motors, solenoids, servos, active actuators, hydraulic actuators, pneumatic actuators, and/or the like that are operated based on signals received from a control unit, such as the control unit 140 of FIG. 1. In some examples, the drive system 340 manipulates a subset of the various DOFs with others of the various DOFs being, for examples, controlled manually by an operator. In some examples, the signals can include one or more currents, voltages, pulse-width modulated wave forms, and/or the like. In some examples, the drive system 340 can include one or more shafts, gears, pulleys, rods, bands, and/or the like which can be coupled to corresponding motors, solenoids, servos, active actuators, hydraulics, pneumatics, and/or the like that are part of the articulated arm, such as any of the repositionable structures 120, to which the instrument 300 is mounted. In some examples, the one or more drive inputs, such as shafts, gears, pulleys, rods, bands, and/or the like, can be used to receive forces and/or torques from the motors, solenoids, servos, active actuators, hydraulics, pneumatics, and/or the like and apply those forces and/or torques to adjust the various DOFs of the instrument 300.

In some embodiments, the forces and/or torques generated by and/or received by drive system 340 are transferred from the drive system 340 and along the shaft 310 to the various joints and/or elements of the instrument 300 located distal to the drive system 340 using one or more drive mechanisms 350. In some examples, the one or more drive mechanisms 350 include one or more gears, levers, pulleys, cables, rods, bands, and/or the like. In some examples, the shaft 310 is hollow and the drive mechanisms 350 pass along the inside of the shaft 310 from the drive system 340 to the corresponding DOF in the end effector 320 and/or the articulated wrist 330. In some examples, each of the drive mechanisms 350 can be a cable disposed inside a hollow sheath or lumen in a Bowden cable like configuration, a shaft or rod whose rotation actuates a corresponding DOF, and/or the like. In some examples, the cable and/or the inside of the lumen can be coated with a low-friction coating such as polytetrafluoroethylene (PTFE) and/or the like. In some examples, as the proximal end of each of the cables is pulled and/or pushed inside the drive system 340, such as by wrapping and/or unwrapping the cable about a capstan or shaft, the distal end of the cable moves accordingly and applies a suitable force and/or torque to adjust one of the DOFs of the end effector 320, the articulated wrist 330, and/or the instrument 300. In some examples, the drive system 340 can be controlled and/or receive instructions from a control module, such as the control module 170.

In some embodiments, the instrument 300 further includes an energy delivery system 360 located at the proximal end of shaft 310. The energy delivery system 360 includes one or more components for generating energy for delivery by the instrument 300 and/or for receiving energy from an energy generation system that is separate from instrument 300. In some examples, the energy can be in one or more energy modalities including ultrasonic, radio frequency, electrical, magnetic, thermal, light, and/or the like. In some examples, energy delivery system 360 provides energy to one or more transducers, signal generators, and/or the like (not shown) that are operated based on signals received from a control unit, such as the control unit 140 of FIG. 1. In some examples, the signals can include one or more currents, voltages, pulse-width modulated wave forms, light patterns, and/or the like.

In some embodiments, the energy generated by and/or received by the energy delivery system 360 is transferred from the energy delivery system 360 and along the shaft 310 to the various joints and/or elements of the instrument 300 located distal to the energy delivery system 360 using one or more energy delivery mechanisms 370. In some examples, the one or more energy delivery mechanisms 370 include one or more wires, cables, optical fibers, and/or like. In some examples, the shaft 310 is hollow and energy delivery mechanisms 370 pass along the inside of shaft 310 from the energy delivery system 360 to the end effector 320 for delivery to a material within the workspace. In some examples, the energy delivery system 360 can be controlled and/or receive instructions from a control module, such as the control module 170.

Typically, control of the drive system 340 and the energy delivery system 360 have been decoupled, with the control module 170 independently controlling each system. Conventional approaches include the control module 170 initially controlling the jaws of the end effector 320 to grasp a material before delivering energy. However, when applying certain types of energy, such as delivering ultrasonic energy as part of a sealing process, the control module 170 does not effectively perform kinematic control via the drive system 340 and energy control via the energy delivery system simultaneously. As a result, such control modules 170 may improperly control the grasp or the delivery of energy, causing errors associated with improper sealing, such as oozing, incomplete seals, and other seal errors.

In view of the foregoing, an improved version of the instrument 300 is controlled by the control module 170 to concurrently monitor and control kinematic parameters (e.g., jaw parameters and grasp parameters) when sealing and cutting a material using ultrasonic energy. In particular, the control module 170 monitors the jaws grasping a material to determine whether the parameters meet a seal threshold before causing the energy delivery system 360 to deliver a seal energy output to the grasped material. The control module 170 monitors the energy delivered to the material and adjusts the energy delivered such that the end effector provides ultrasonic energy at the resonant frequency as the resonant frequency changes during the sealing process. Upon determining that a seal is complete, the control module 170 causes the energy delivery system 360 to a cut energy output (e.g., a higher energy output than the seal energy output) to cut the sealed material.

In this manner, the control module 170 can control the instrument 300 to provide a more accurate power output to seal and cut a grasped material. Further, by monitoring kinematic parameters and energy parameters from the instrument 300 during the sealing and cutting process, the control module 170 can provide feedback to the operator indicating the success or failure of a sealing and cutting procedure.

In various embodiments, the instrument 300 includes one or more transducers distal to the energy delivery system 360 that converts the energy received from the energy delivery system into another form of energy. In one example, the end effector 320 includes one or more transducers included in at least one of the jaws of or located proximal to the jaws. The transducers receive an electronic signal and generate a vibration at a corresponding amplitude, frequency, and phase. The transducers vibrate while in contact with the grasped material, generating friction between the jaws and the grasped material, heating the material in a manner that causes sealing (in a first range of energy levels) and/or cutting in a second range of energy levels). In some examples, the energy levels for sealing and/or cutting are selected based on the material to be cut and/or sealed. In some examples, when the material is anatomical tissue, power levels in a range of about 5 to 20 W and/or current levels of 80 to 150 mA can generally cause sealing, and power levels in a range of about 10 to 40 W can and/or current levels of 100 to 200 mA generally cause cutting. In some examples, power levels and/or current levels between the levels for sealing and the levels for cutting result in a combination of sealing and cutting. Alternatively, in some embodiments, the transducers can receive energy for concurrent sealing and cutting as part of a synchronous sealing and cutting operation.

In some examples, the control module 170 receives feedback from the transducers (e.g., voltage and current received by the transducer) and determines the impedance of the transducer. In some examples, the end effectors 320, the drive system 340, and/or the energy delivery system 360 include one or more sensors that acquire sensor data about the instrument 300 and/or the grasped material. In some examples, the sensors include sensors that acquire sensor data indicating jaw characteristics (e.g., jaw angle, jaw separation, etc.) and/or material characteristics (e.g., material thickness). In some examples, sensors, such as electrical sensors (e.g., a current sensor, a voltage sensor, etc.) included along the drive system 340, acquires sensor data regarding the impedance of the transducers. In various embodiments, a control module, such as the control module 170 processes the sensor data to determine jaw characteristics of the jaws and/or the transducers, and/or material characteristics of the grasped material to determine the energy to apply to the grasped material and determine whether a seal and/or cut is complete. In some examples, the control module 170 determines the resonant frequency for delivering the ultrasonic energy to the material, where the impedance of the transducer is at a minimum when delivering the ultrasonic energy at resonance. The control module 170 adjusts one or more energy parameters to ensure that the energy delivery system 360 provides energy to the transducer at resonance (e.g., at the resonant frequency and in-phase).

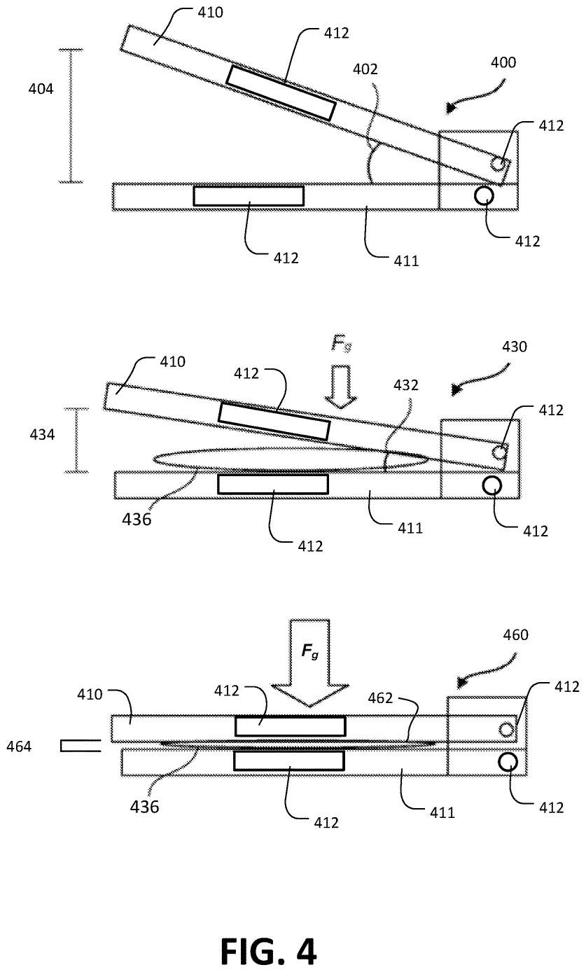

FIG. 4 depicts an effector having a first and second jaw and illustrate jaw characteristics in accordance with one or more embodiments. As shown in view 400, the jaws 410 and 411 of an end effector include one or more transducers 412. In some examples, the jaws 410 and 411 and/or transducers 412 have a set of jaw characteristics. The jaw characteristics includes a separation distance 404, which is a distance between the tips of the jaws 410 and 411. The jaw characteristics can additionally and/or alternatively include a jaw angle 432 between the jaws 410 and 411.

The view 430 illustrates jaws 410 and 411 grasping a material 436 between the jaws 410 and 411 at a grasping force F_{g}. In some examples, a control module, such as the control module 170 determines the torque associated with the moving jaw 410 closing to grasp the material 436 between the moving jaw 410 (e.g., the upper jaw) and a stationary jaw 411 (e.g., the lower jaw). In some examples, the control module 170 measures the jaw characteristics (e.g., the jaw angle 432, separation distance 434, voltage and/or current from the transducers 412, etc.) and/or the material characteristics of the material 436 when the material 436 is grasped between the jaws at the grasping force F_{g}. In response, the control module 170 provides an indication on the user interface (e.g., a user interface displayed on the display unit 254) as to whether the jaw characteristics and/or the material characteristics are appropriate to start sealing and/or cutting operations. For example, in addition to jaw characteristics indicating the jaw angle 432 and/or the jaw separation 434, the material characteristics can include a type of the material 436, such as a body tissue (e.g., bowel, stomach), a thickness of the material 436, a target grasping force Ft, and/or a target jaw angle.

The view 460 illustrates the jaws 410 and 411 having successfully grasped the material 436 between the jaws 410 and 411 and the jaws 410 and 411 have achieved a minimal separation distance 464, a minimal jaw angle 462, and or the like. In some examples, the control module 170 determines jaw characteristics that indicate that sealing and/or cutting can be performed. Such jaw characteristics can include an applied force and/or torque, a rate of change in applied force and/or torque as applied by the jaws to the grasped material (obtained from one or more force and/or torque sensors associated with the jaws, the one or more actuators used to actuate the jaws, and/or the one or more transducers 412). In some examples, the rate of change in applied force and/or torque can be determined using a numerical differentiation technique (e.g., using the divided difference method) from two or more force and/or torque readings obtained over time. In some examples, the force and/or torque can be determined based on one or more currents used to actuate the one or more actuators used to actuate one or both of the jaws 410 and 411.

Once the material 436 is successfully grasped, energy is delivered to the jaws 410 and 411 using the transducers 412 to being the sealing and/or cutting. In some examples, the control module 170 monitors the current and/or voltage of the transducers 412 during the sealing and/or sealing operation to determine a resonant frequency for the transducers 412. In such instances, the control module 170 can adjust the frequency of the current and/or voltage delivered to the transducers 412 to ensure that the transducers 412 are delivering energy at resonance to improve the effectiveness of the energy delivery.

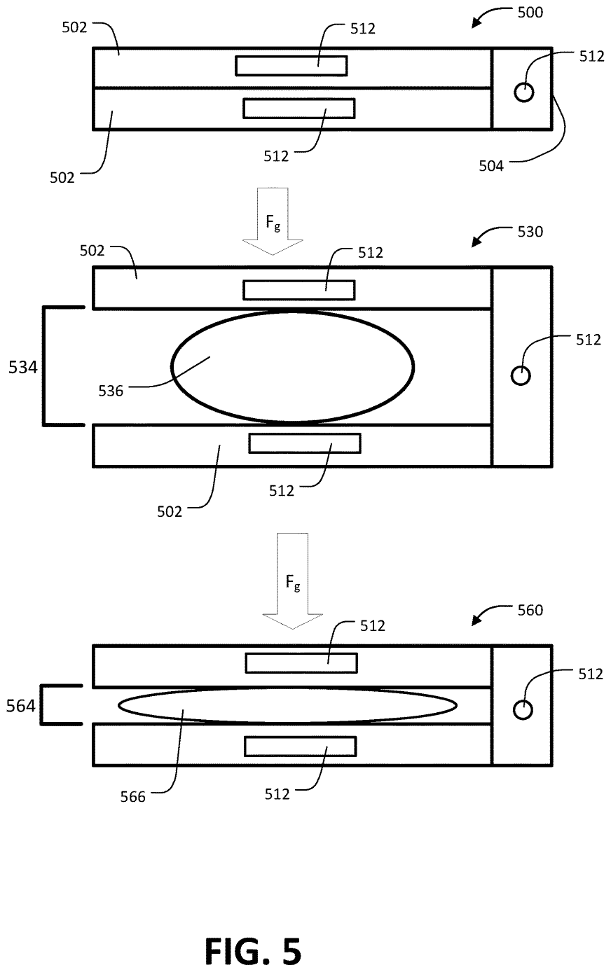

FIG. 5 depicts another effector having a first and second jaw and illustrate the jaw characteristics in accordance with one or more embodiments. As shown in view 500, the jaws 502 of an end effector are connected via a linear track 504.

The view 530 illustrates the jaws 502 grasping a material 536 between the jaws at a grasping force F_{g}. In some examples, the control module 170 measures the jaw characteristics (e.g., the separation distance 534, etc.) and/or the material characteristics when the material 536 is grasped between the jaws 502 at the grasping force F_{g}. In response, the control module 170 provides an indication on the user interface as to whether the jaw characteristics and/or the material characteristics are appropriate for the sealing and/or cutting operations.

The view 560 illustrates the jaws having successfully grasped the material 566 between the jaws 502 and the jaws 502 have achieved a minimal separation distance 534 and or the like. In some examples, the control module 170 determines jaw characteristics that indicate that sealing and/or cutting can be performed. Such jaw characteristics can include an applied force, a rate of change in applied force as applied by the jaws to the grasped material (obtained from one or more force sensors associated with the jaws and/or the one or more actuators used to actuate the jaws). In some examples, the rate of change in applied force can be determined using a numerical differentiation technique (e.g., using the divided difference method) from two or more force readings obtained over time. In some examples, the force can be determined based on one or more currents used to actuate the one or more actuators used to actuate one or both of the jaws.

Once the material 536 is successfully grasped, energy is delivered to the jaws 502 using the transducers 512 to being the sealing and/or cutting. In some examples, the control module 170 monitors the current and/or voltage of the transducers 512 during the sealing and/or sealing operation to determine a resonant frequency for the transducers 512. In such instances, the control module 170 can adjust the frequency of current and/or voltage delivered to the transducers 512 to ensure that the transducers 512 are delivering energy at resonance to improve the effectiveness of the energy delivery.

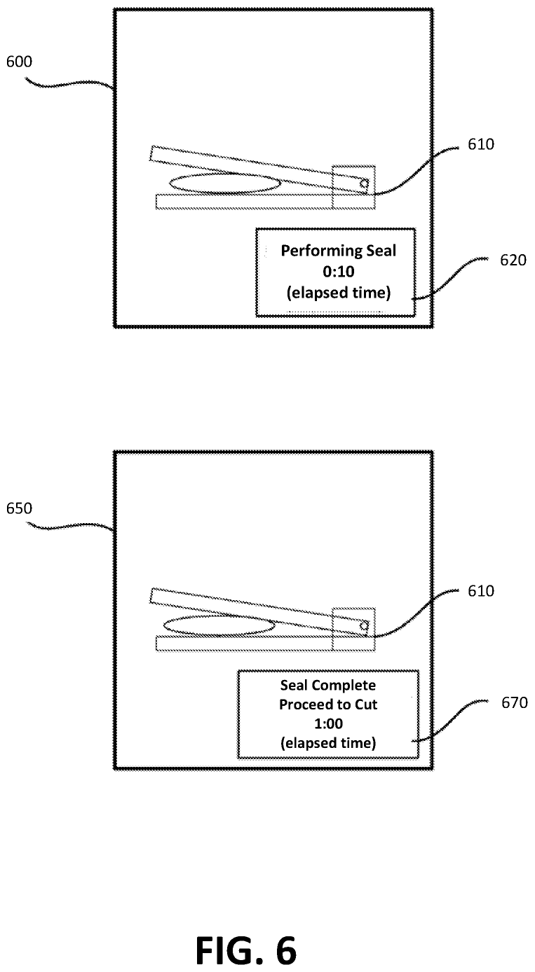

FIG. 6 illustrates a user interface 600 having an indicator 620, 670 of sealing status in accordance with one or more embodiments. In some examples, the user interface 600 is displayed in a display unit, such as the display unit 254. As shown, the user interface 600 depicts a view of jaws grasping a material. In some examples, the user interface 600 displays images and/or visual representations of the end effectors during a procedure in addition to the indicators of an activation status for sealing and/or cutting energy delivery (e.g., performing sealing operation, abort operation, indicating that operation is complete, etc.). A control module, such as the control module 170, superimposes the indication of activation status over the images on the user interface 600 during the surgical procedure so as to seamlessly incorporate the feature into the surgical procedure. As shown, the user interface 600 includes an illustration 610 of the jaws performing the grasping operation and the indicator 620 superimposed on the lower right area of the user interface 600, where the indicator 620 indicates that sealing is being performed and the elapsed time since activation.

The display 650 illustrates a later time during operation. As shown, the user interface 600 includes an illustration 610 of the jaws performing the sealing operation and the indicator 620 superimposed on the lower right area of the user interface 600, where the indicator 670 indicates that sealing is complete and the elapsed time since activation. In some examples, the control module 170 updates the indicator 670 to indicate that the sealing operation is complete and that the operator is free to perform a cutting operation.

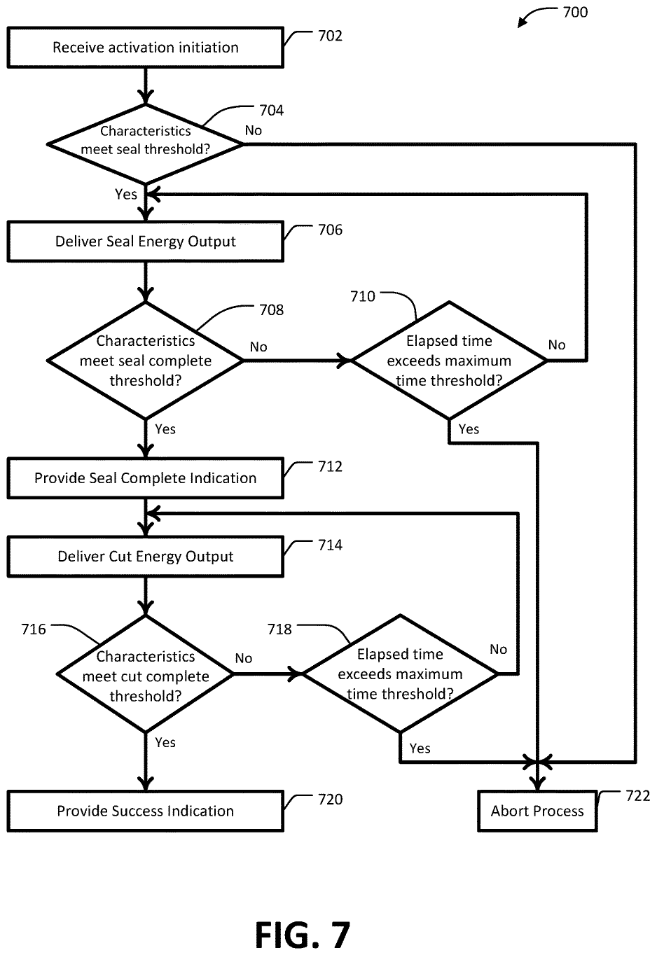

FIG. 7 is a flow diagram of a method 700 for ultrasonic energy delivery in accordance with one or more embodiments. Although the method steps are described in conjunction with the systems of FIGS. 1-3 and the examples of FIGS. 4-6, persons of ordinary skill in the art will understand that any system configured to perform the method steps, in any order, is within the scope of the present disclosure. One or more of the processes 702-722 of the method 700 can be implemented, at least in part, in the form of executable code stored on non-transient, tangible, machine-readable media. This executable code, when executed by a processor (e.g., the processing system 150 in the control unit 140), can cause the processor to perform one or more of the processes 702-722. In some embodiments, the method 700 can be performed by a module, such as the control module 170. In some embodiments, the method 700 can be used to apply ultrasonic energy to a material using an instrument. The ultrasonic energy can apply a first energy to seal a material and, upon determining that the seal is complete, delivers a second energy to cut the material.

Aspects of the method 700 are described via reference to FIG. 4, which illustrates movement of one or more jaws to apply force and/or torque to grasp a material for sealing and/or cutting in accordance with one or more embodiments. Further, aspects of the method 700 are described via reference to FIG. 5, which illustrates movement of one or more joints to apply a force to grasp a material for sealing and/or cutting in accordance with one or more embodiments. Further, aspects of the method 700 are described via reference to FIG. 6, which illustrate indications of providing a seal complete indication, in accordance with one or more embodiments. However, it is understood that the examples of FIGS. 4-6 are not restrictive, and that other values, shapes, behaviors, and/or the like depicted in FIGS. 4-6 can be different for different input controls, different repositionable structures, different follower instruments, different DOFs, different procedures, different viewable objects, and/or the like.

At a process 702, a control module, such as control module 170, receives an initiation of an activation process to cut and seal a material grasped between jaws of an instrument. In some examples, the control module receives an input via the user input system 250, such as an input received via a button, switch, pedal, lever, user interface, voice command, gesture command, and/or the like. In some examples, control module 170 receives the initiation when an instrument, such as the instrument 300 grasps a material. The material can be grasped between the jaws of an end effector, such as the end effector 320. In some examples, each of the jaws can be consistent with the jaw 410, 411 or 502. In some examples, the material can be grasped using a drive system, such as drive system 340, under the control of the control module 170. In some examples, the control module 170 provides an indication, such as a tone or other output (e.g., a visual indicator, voice status, haptic feedback, etc.) to indicate the change in activation status.

At a process 704, the control module 170 determines whether characteristics associated with the grasped material meet a seal threshold. In various embodiments, the seal threshold is associated with characteristics of the grasping that are associated with an increased probability of a successful seal operation. In some examples, the seal threshold can be based on one or more jaw characteristics and/or material characteristics, such as jaw angle, jaw separation, grasp force, and/or torque. For example, the seal threshold can correspond to the jaws reaching a desired angle (e.g., the jaw angle 462) between the jaws is reached, a desired separation between the jaws is reached (e.g., whether the jaw separation 564 has met a separation distance threshold), and/or a desired force or torque limit indicating a desired grasp strength is reached. In some examples, the grasp can actuate the jaws to a desired position set point (e.g., the desired angle and/or separation between the jaws) subject to an upper force and/or torque limit. In some examples, the control module 170 determines the jaw characteristics and/or material characteristics using absolute measurements (e.g., a specific angle for the jaw angle). In some examples, the control module 170 determines the jaw characteristics and/or material characteristics using proportional changes (e.g., change in jaw angle, change in jaw separation, change in material impedance, etc.).

In some examples, the control module 170 acquires the jaw characteristics and/or material characteristics before determining whether the characteristics meet the seal threshold. In some examples, various measurements are acquired via sensors on an instrument, such as the instrument 300. In some examples, the jaw characteristics, such the applied pressure, can be determined using one or more pressure sensors (e.g., one or more strain gauges, pressure transducers, pressure sensitive fiber optic sensors, and/or the like) located along the face of one or both of the jaws. In some examples, a rate of change in applied pressure can be determined using a numerical differentiation technique (e.g., using the divided difference method) from two or more applied pressure readings obtained over time. In some examples, the applied pressure can be determined indirectly from one or more other characteristics. In some examples, the one or more jaw characteristics can include additional kinematic information associated with the instrument and/or the end effector whose jaws are used to grasp the material. In some examples, the additional kinematic information can include information about an amount and/or a type of articulation of an articulated wrist (e.g., articulated wrist 330) of the instrument.

In some examples, the one or more grasp characteristics can be determined from one or more images obtained from an imaging device capturing images of the jaws and the grasped material. In some examples, the one or more images can be used to measure jaw angle, jaw separation, and/or wrist articulation. In some examples, the imaging device can be an endoscope and/or a stereoscopic endoscope. In some examples, the imaging device can be mounted as an instrument to a repositionable arm, such as one of the one or more repositionable structures 120.

In some examples, the one or more material characteristics can include a desiccation level (e.g., moisture content) of the grasped material. In some examples, the desiccation level can provide an indication of whether the material is ready for cutting and/or sealing (e.g., it can be advantageous to squeeze moisture out the material by grasping before cutting and/or sealing). In some examples, the desiccation level can be determined from the jaw angle and/or separation, the applied force and/or torque, the applied pressure, the stiffness, and/or the temperature for the grasped material. In some examples, the control module 170 can compute one or more formulas and/or refer to one or more look-up tables, non-linear maps, and/or the like to determine the desiccation level of the grasped material from the jaw angle and/or separation, the applied force and/or torque, the applied pressure, the stiffness, and/or the temperature.

In some examples, the control module 170 modifies the seal thresholds based on initial measurements. In some examples, the control module 170 acquires measurements of material thickness. Based on the material thickness, the control module 170 modifies the seal threshold, such as modifying the raising the change in material thickness upon measuring an above-average initial thickness of the grasped material.

When the control module 170 determines that the characteristics meet the seal threshold, the control module proceeds to process 706. Otherwise, the control module 170 determines that the characteristics do not meet the seal threshold and proceeds to process 722.

At a process 706, the control module delivers a seal energy output. In some examples, the control module 170 controls the energy delivered by the instrument based on the one or more characteristics determined during process 704. In some examples, the control module 170 delivers ultrasonic energy via an energy delivery system (e.g., the energy delivery system 360) and transducers (e.g., the transducers 412 and/or 512) included in the instrument to vibrate the one or more jaws at a frequency, amplitude, and phase controlled by the control module 170 and create friction with the grasped material, which heats the material and causes sealing of the material.

In some examples, the one or more characteristics can be applied as inputs to the control module 170 to determine one or more control parameters for controlling the energy delivery to the material. The control module 170 uses the control parameters to control the grasp and/or the energy delivery of the instrument. In some examples, the control modules 170 uses one or more control algorithms based on the control parameters to provide one or more commands, signals, and/or the like to the systems for grasp and energy delivery, such as the drive system 340 and/or the energy delivery system 360.

In some examples, the one or more control parameters can include one or more of a grasp set point (e.g., a grasp angle and/or separation), a rate of change in grasp set point (e.g., a grasping velocity), a force and/or torque set point, a force or torque set point, a current set point for one or more actuators used to actuate the jaws, a resonant frequency at which the one or more transducers vibrate, a pressure set point, a target set point for the impedance of the one or more transducers, an amount of sealing energy delivered to the grasped material, and/or the like.

In some examples, the control module 170 determines that the amount of energy to deliver based on the jaw angle and/or separation. In some examples, when the jaw angle and/or separation is larger, more energy is delivered because more material is grasped, and when the jaw angle and/or separation is smaller, less energy is delivered because less material is grasped. In some examples, the relationship between the jaw angle and/or separation and energy to deliver can be one or more of linear, monotonic, subject to maximum and minimum energy delivery limits, and/or the like. In some examples, the control module 170 controls the amount of energy to deliver in an inverse relationship to the rate of change in jaw angle and/or separation. In some examples, when the rate of change in jaw angle and/or separation is smaller, more energy is delivered to address stiffer and/or more slowly desiccating material and when the rate of change in jaw angle and/or separation is larger, less energy is delivered to address less stiff and/or more rapidly desiccating material. In some examples, the relationship between the rate of change jaw angle and/or separation and energy to deliver can be monotonic, subject to maximum and minimum energy delivery limits, and/or the like.

In some examples, the control module 170 generates a handshake signal to the energy delivery system 360 to transmit electrical parameters to the transducers to provide the applicable energy output during the sealing operation. In some examples, the control module 170 modifies the electrical parameters to change the energy output based on initial measurements. For example, upon measuring the material thickness, the control module increases or reduces the ultrasonic energy that the transducers provide to seal the grasped material. In such instances, thinner or less vascular tissue uses a lower energy output, while thicker or more vascular tissue uses a larger energy output.

At a process 708, the control module 170 determines whether characteristics associated with sealing the material meet a seal complete threshold. In various embodiments, the control module 170 maintains a seal complete threshold for finishing a seal operation, where the seal complete threshold is associated with the jaw characteristics and/or material characteristics indicating a high likelihood of a successfully completed seal operation. In some embodiments, the control module 170 maintains a set of seal complete thresholds. In some examples, the control module 170 determines that a seal is completed when two or more of the set of seal complete thresholds have been met.

In some examples, the seal complete threshold can be based on one or more jaw characteristics and/or material characteristics, such as jaw angle, jaw separation, grasp force, torque, impendence of the transducers, change in impedance (e.g., a change in amplitude, frequency, and/or phase angle received by the transducers), resonant frequency detected by the transducer, and/or change in the resonant frequency (e.g., whether the detected resonant frequency has changed beyond a predetermined resonant frequency threshold). In some examples, the seal complete threshold can be based on a desired angle between the jaws (e.g., a jaw angle threshold), a desired separation between the jaws (e.g., a separation distance threshold), a desired force or torque, a desired resonant frequency of the transducers and/or a change in jaw angle separation, force, and/or torque. In some examples, the seal complete threshold can be based on a predetermined resonant frequency threshold, where the control module 170 determines that a seal is complete when the resonant frequency is below the resonant frequency threshold.

In some examples, the control module 170 determines the jaw characteristics and/or material characteristics using absolute measurements (e.g., a specific angle for the jaw angle). In some examples, the control module 170 determines the jaw characteristics and/or material characteristics using proportional changes (e.g., change in resonant frequency of the transducers, change in the impedance of the transducers, etc.). For example, the control module 170 can determine whether the change in jaw angle changed (e.g., got smaller) in a range of 20% to 50% compared to the jaw angle at the start of activation. In another example, the control module 170 determines whether a change in electrical impedance of the transducers and/or change in the resonance frequency at the transducers has changed within a specified range or has changed beyond a specific threshold.

In some examples, the control module 170 acquires one or more measurements of material thickness during activation and/or during the sealing process. Based on the one or more measurements of the material thickness, the control module 170 modifies the seal complete threshold to correspond to the one or more measurements.

According to some embodiments, the control module 170 receives as input any of the one or more characteristics determined during process 704 and determines one or more parameters that correspond to a seal complete condition. In some examples, the one or more parameters that correspond to the seal complete condition can include a threshold impedance of the transducers that indicates that sufficient energy has been delivered to the grasped material for a successful sealing. In some examples, the threshold impedance can correspond to a minimum impedance that should be reached before energy delivery is complete.

When the control module 170 determines that the characteristics meet the seal complete threshold, the control module proceeds to process 712. Otherwise, the control module 170 determines that the characteristics do not meet the seal complete threshold and proceeds to process 710.

At a process 710, the control module 170 determines whether the elapsed time for the sealing process exceeds a maximum time threshold for the sealing process. In some examples, the control module 170 compares the elapsed time that the instrument has delivered a seal energy output to a sealing time threshold corresponding to a maximum time threshold that control module 170 has associated with a successful seal operation. In such instances, the control module 170 aborts the sealing process if the elapsed time exceeds the maximum threshold for sealing. In some examples, the control module 170 modifies the maximum time threshold and/or amount of energy to deliver based on one or more of a type of the material, operator preference, elapsed time, and/or the like. In some examples, thinner or less vascular tissue have shortened seal times, while thicker or more vascular tissue have longer seal times. The control module 170 can respond to the material thickness by modifying the maximum time threshold for sealing.

When the control module 170 determines that the elapsed time has not exceeded the maximum time threshold, the control module 170 returns to process 706. Otherwise, the control module 170 determines the elapsed time has exceeded the maximum time threshold, the control module 170 and proceeds to process 722. In some examples, the control module 170 performs process 706 by adjusting the frequency of the seal energy output to maintain energy delivery at the resonance frequency. In some examples, the control module 170 determines the impedance of the transducers, where the impedance is at a minimum when the transducers deliver ultrasonic energy at resonance. In another example, the control module 170 can determine the resonant frequency by determining whether the transducer is delivering energy in phase.

At a process 712, the control module 170 provides a seal complete indication. In some examples, the control module 170 causes the control unit 140 and/or user input system 250 to provide an indication to the operator that the seal process has been successfully completed. In some examples, the indication is an audio tone corresponding to a seal complete determination. In some examples, the indication includes one or more types of outputs (e.g., a visual indicator, voice status, haptic feedback, etc.) that indicate a change in activation status. In some examples, a user interface, such as the user interface 600 includes the indicator 670 that specifies the activation status. In some examples, the seal complete indication also indicates a change in activation status from a sealing process to a cutting process.

At a process 714, the control module 170 delivers a cut energy output. In some examples, the control module 170 controls the energy delivered by the instrument via the transducers based on the one or more characteristics determined during process 704 and/or process 708. In some examples, the control module 170 delivers ultrasonic energy via the energy delivery system 360 and transducers 412 and/or 512 included in the instrument 300 to vibrate the one or more jaws and create friction with the material, generating that is sufficient to cut the material.

In some examples, the control module 170 processes the one or more characteristics to determine one or more control parameters for controlling the energy delivery to the material via the transducers during the cutting process. The control module 170 uses the control parameters to control the grasp and/or the energy delivery of the instrument during the cutting process. In some examples, the control module 170 provides one or more commands, signals, and/or the like to the systems for grasp and energy delivery, such as the drive system 340 to maintain the grasp of the material and/or the energy delivery system 360 via the transducers to cut the material.

In some examples, the control module 170 generates a handshake signal to the energy delivery system 360 to transmit electrical parameters to the transducers to provide the applicable energy output during the cutting operation. In some examples, the control module 170 modifies the electrical parameters to change the energy output based on initial measurements. For example, upon measuring the material thickness, the control module increases or reduces the ultrasonic energy that the transducers provide to cut the grasped material. In such instances, thinner or less vascular tissue uses a lower energy output, while thicker or more vascular tissue uses a larger energy output.

At a process 716, the control module 170 determines whether characteristics associated with cutting the material meet a cut complete threshold. In various embodiments, the control module 170 maintains a cut complete threshold for finishing a cutting operation, where the cut complete threshold is associated with the jaw characteristics and/or material characteristics indicating a high likelihood of a successfully completed cut operation. In some examples, the control module 170 maintains a set of cut complete thresholds. In some examples, the control module 170 determines that a cut is completed when two or more of the set of cut complete thresholds have been met.

In some examples, the cut complete threshold can be based on one or more jaw characteristics and/or material characteristics, such as jaw angle, jaw separation, grasp force, torque, impendence of the transducers providing the ultrasonic energy, change in impedance of the transducers, resonance frequency of the energy delivery (e.g., a change in amplitude, frequency, and/or phase angle of 20% to 50%), and/or change in the resonance frequency. In some examples, the seal complete threshold can be based on a desired angle between the jaws is reached (e.g., a jaw angle threshold), a desired separation between the jaws is reached (e.g., a separation distance threshold), a desired force or torque, and/or a change in jaw angle separation, force, and/or torque.

In some examples, the control module 170 determines the jaw characteristics and/or material characteristics using absolute measurements (e.g., a specific angle for the jaw angle). In some examples, the control module 170 determines the jaw characteristics and/or material characteristics using proportional changes (e.g., change in resonant frequency, change in material impedance, etc.). For example, the control module 170 can determine whether the change in jaw angle changed in a range of 80% to 100% compared to the jaw angle at the start of activation. In another example, the control module 170 can determine whether a change in electrical impedance characteristics and/or change in the resonance frequency applied by the transducers has changed within a specified range.

In some examples, the control module 170 acquires one or more measurements of material thickness during activation and/or during the sealing process. Based on the one or more measurements of the material thickness, the control module 170 modifies the cut complete threshold to correspond to the one or more measurements. According to some embodiments, the control module 170 determines a cut complete condition indicating when the energy delivery for sealing is complete. In some examples, the control module 170 receives as input any of the one or more characteristics determined during process 714 and determines one or more parameters that correspond to a cut complete condition. In some examples, the one or more parameters that correspond to the cut complete condition can include a threshold impedance of the transducers indicating that the cut is complete.

When the control module 170 determines that the characteristics meet the cut complete threshold, the control module 170 proceeds to process 720. Otherwise, the control module 170 determines that the characteristics do not meet the seal complete threshold and proceeds to process 718.

At a process 718, the control module 170 determines whether the elapsed time for the cutting process exceeds a maximum time threshold for the cutting process. In some examples, the control module 170 compares the elapsed time that the instrument has delivered a cut energy output to a cut time threshold corresponding to a maximum time threshold that the control module 170 has associated with a successful cut operation. In such instances, the control module 170 aborts the cutting operation if the elapsed time exceeds the maximum time threshold for cutting. In some examples, the control module 170 modifies the maximum time threshold and/or amount of energy to deliver based on one or more of a type of the material, operator preference, elapsed time, and/or the like. In some examples, as thinner or less vascular tissue have shortened cutting times, while thicker or more vascular tissue have longer cutting times. The control module 170 can respond to the material thickness by modifying the maximum time threshold for cutting.

When the control module determines that the elapsed time has not exceeded the maximum time threshold, the control module 170 returns to process 714. Otherwise, the control module 170 determines the elapsed time has exceeded the maximum time threshold, the control module 170 proceeds to process 722.

At a process 720, the control module 170 provides a process complete indication. In some examples, the control module 170 causes the control module 170 and/or user input system 250 to provide an indication to the operator that the synchronous sealing and cutting process has been successfully completed. In some examples, the indication is an audio tone corresponding to a seal complete determination. In examples, the indication includes one or more types of outputs (e.g., a visual indicator, voice status, haptic feedback, etc.) that indicate a change in activation status. In some examples, a user interface, such as the user interface 600 includes the indicator 670 that specifies the activation status. In some examples, the cut complete indication also indicates a change in activation status to indicate a completion of the synchronous sealing and cutting process.

At a process 722, the control module 170 provides an abort indication. In some examples, the control module 170 causes the instrument to stop delivering energy (e.g., stop the transducers from delivering seal energy and/or cut energy) via the energy delivery system 360. In some examples, the control unit 140 and/or user input system 250 to provide an indication to the user that a part of the synchronous sealing and cutting process has been unsuccessful. In some examples, an alert and/or notification can be provided to the operator indicating that cutting and/or sealing was not successfully completed due to the expiration of a maximum time period associated with the sealing process and/or the cutting process. In examples, the indication includes one or more types of outputs (e.g., a visual indicator, voice status, haptic feedback, etc.) that indicate a change in activation status.

Some examples of control systems, such as control unit 140 can include non-transitory, tangible, machine readable media that include executable code that when executed by one or more processors (e.g., a processor in the processing system 150) can cause the one or more processors to perform the processes of method 700 and/or the processes of FIG. 7. Some common forms of machine readable media that can include the processes of method 700 and/or the processes of FIG. 7 are, for example, floppy disk, flexible disk, hard disk, magnetic tape, any other magnetic medium, CD-ROM, any other optical medium, punch cards, paper tape, any other physical medium with patterns of holes, RAM, PROM, EPROM, FLASH-EPROM, any other memory chip or cartridge, and/or any other medium from which a processor or computer is adapted to read.

Although illustrative embodiments have been shown and described, a wide range of modification, change and substitution is contemplated in the foregoing disclosure and in some instances, some features of the embodiments can be employed without a corresponding use of other features. One of ordinary skill in the art would recognize many variations, alternatives, and modifications. Thus, the scope of the invention should be limited only by the following claims, and it is appropriate that the claims be construed broadly and, in a manner, consistent with the scope of the embodiments disclosed herein.

1. A computer-assisted device comprising:
an end effector having a first jaw, a second jaw, and at least one transducer for delivering ultrasonic energy using at least one of the first jaw or the second jaw; and
a processing system coupled to the end effector, the processing system being configured to:
control the first jaw and the second jaw to grasp a material;
determine one or more first characteristics of the grasping;
control a first energy delivered by the at least one transducer to seal the material based on the one or more first characteristics;
determine one or more second characteristics associated with the first energy delivered by the at least one transducer; and
in response to completing sealing of the material, control at least a second energy delivered by the at least one transducer to cut the grasped material based on the one or more first characteristics and the one or more second characteristics associated with the first energy delivered.

2. The computer-assisted device of claim 1, wherein the one or more first characteristics of the grasping includes one or more of:
a jaw angle formed between the first jaw and the second jaw;
a separation distance between a first portion of the first jaw and a second portion of the second jaw; or
a grasp force or torque asserted by the first jaw and the second jaw on the material.

3. The computer-assisted device of claim 1, wherein to determine whether sealing of the material is complete, the processing system is configured to:
determine one or more third characteristics of the grasping; and
end delivery of the first energy in response to the one or more third characteristics of the grasping reaching a predetermined percentage of the one or more first characteristics of the grasping.

4. The computer-assisted device of claim 1, wherein to determine whether sealing of the material is complete, the processing system is configured to:
determine that a resonant frequency of the at least one transducer when delivering the first energy has changed beyond a predetermined resonant frequency threshold.

5. The computer-assisted device of claim 1, wherein the processing system is configured to:
determine that a resonant frequency of the at least one transducer when delivering the second energy has changed beyond a predetermined resonant frequency threshold; and
end delivery of the second energy by the at least one transducer.

6. The computer-assisted device of claim 1, wherein the at least one transducer is included in the first jaw or the second jaw.

7. The computer-assisted device of claim 1, wherein the at least one transducer is located proximal to at least one of the first jaw or the second jaw.

8. The computer-assisted device of claim 1, wherein to control the first energy or the second energy delivered by the at least one transducer, the processing system is configured to keep the at least one transducer vibrating at a resonance frequency.

9. (canceled)

10. The computer-assisted device of claim 1, wherein the processing system is further configured to determine one or more characteristics of the material, wherein control of the first energy is further based on the one or more characteristics of the material.

11. (canceled)

12. The computer-assisted device of claim 1, wherein the one or more second characteristics associated with the first energy delivered include at least one of:
a jaw angle formed between the first jaw and the second jaw;
a separation distance between a first portion of the first jaw and a second portion of the second jaw;
a resonant frequency of the end effector and the material, obtained by the at least one transducer; or
a grasp force or torque asserted by the first jaw and the second jaw on the material.

13-15. (canceled)

16. The computer-assisted device of claim 1, wherein the processing system is further configured to end the first energy or the second energy delivered by the at least one transducer when a separation distance between the first jaw and the second jaw falls below a predetermined separation distance threshold or a jaw angle between the first jaw and the second jaw falls below a predetermined jaw angle threshold.

17-19. (canceled)

20. A method comprising:
controlling, by a processing system of a computer-assisted device, a first jaw and a second jaw of an end effector to grasp a material;
determining, by the processing system, one or more first characteristics of the grasping;
controlling, by the processing system, a first energy delivered by at least one transducer to seal the material based on the one or more first characteristics, the at least one transducer delivering ultrasonic energy using at least one of the first jaw or the second jaw;
determining one or more second characteristics associated with the first energy delivered by the at least one transducer; and
in response to completing sealing of the material, controlling, by the processing system, at least a second energy delivered by the at least one transducer to cut the grasped material based on the one or more first characteristics and the one or more second characteristics.

21. The method of claim 20, wherein the one or more first characteristics of the grasping includes one or more of:
a jaw angle formed between the first jaw and the second jaw;
a separation distance between a first portion of the first jaw and a second portion of the second jaw; or
a grasp force or torque asserted by the first jaw and the second jaw on the material.

22. The method of claim 20, wherein determining whether sealing of the material is complete comprises:
determining one or more third characteristics of the grasping; and
ending delivery of the first energy in response to the one or more third characteristics of the grasping reaching a predetermined percentage of the one or more first characteristics of the grasping.

23. The method of claim 20, wherein determining whether sealing of the material is complete comprises:
determining that a resonant frequency of the at least one transducer when delivering the first energy has changed beyond a predetermined resonant frequency threshold.

24. The method of claim 20, further comprising:
determining, by the processing system, that a resonant frequency of the at least one transducer when delivering the second energy has changed beyond a predetermined resonant frequency threshold; and
ending, by the processing system, delivery of the second energy by the at least one transducer.

25-26. (canceled)

27. The method of claim 20, wherein controlling the first energy delivered or controlling the second energy delivered by the at least one transducer comprises keeping the at least one transducer vibrating at a resonance frequency.

28-30. (canceled)

31. The method of claim 20, wherein the one or more second characteristics associated with the first energy delivered include at least one of:
a jaw angle formed between the first jaw and the second jaw;
a separation distance between a first portion of the first jaw and a second portion of the second jaw;
a resonant frequency of the end effector and the material, obtained by the at least one transducer; or
a grasp force or torque asserted by the first jaw and the second jaw on the material.

32-38. (canceled)

39. A non-transitory machine-readable medium comprising a plurality of machine-readable instructions which when executed by one or more processors associated with a computer-assisted device, are adapted to cause the computer-assisted device to perform a method comprising:
controlling, by a processing system of a computer-assisted device, a first jaw and a second jaw of an end effector to grasp a material;
determining, by the processing system, one or more first characteristics of the grasping;
controlling, by the processing system, a first energy delivered by at least one transducer to seal the material based on the one or more first characteristics, the at least one transducer delivering ultrasonic energy using at least one of the first jaw or the second jaw;
determining one or more second characteristics associated with the first energy delivered by the at least one transducer; and
in response to completing sealing of the material, controlling, by the processing system, at least a second energy delivered by the at least one transducer to cut the grasped material based on the one or more first characteristics and the one or more second characteristics.

40. The non-transitory machine-readable medium of claim 39, wherein the one or more second characteristics associated with the first energy delivered include at least one of:
a jaw angle formed between the first jaw and the second jaw;
a separation distance between a first portion of the first jaw and a second portion of the second jaw;
a resonant frequency of the end effector and the material, obtained by the at least one transducer; or
a grasp force or torque asserted by the first jaw and the second jaw on the material.
